(19) United States
(12) Patent Application Publication
(10) Pub. No.: US 2026/0354787 A1
(43) Pub. Date: Nov. 12, 2026

(54) ULTRASOUND MICROVASCULAR IMAGING METHOD BASED ON RANK DECOMPOSITION FILTERING

(71) Applicant: ESONIC MEDICAL TECHNOLOGY (BEIJING) CO., LTD., Beijing (CN)

(72) Inventors: Zhenyao Wen, Beijing (CN); Ming Sun, Beijing (CN); Tengfei Shi, Beijing (CN); Jacques Souquet, Beijing (CN)

(21) Appl. No.: 19/119,796

(22) PCT Filed: Jul. 1, 2024
(86) PCT No.: PCT/CN2024/102979

§ 371 Date: Apr. 21, 2026

(30) Foreign Application Priority Data
Aug. 1, 2023 (CN) 202310958805.6

(51) Int. Cl. A61B 8/06; A61B 8/00

(52) U.S. Cl. CPC A61B 8/06; A61B 8/488; A61B 8/5207; A61B 8/5223; A61B 8/5269

(57) ABSTRACT

The present disclosure provides an ultrasonic micro blood flow imaging method based on rank decomposition filtering, including: step one, acquiring ultrasonic ultrafast plane wave data; step two, reading the amount of decomposed rank-1 tensors and performing a CP decomposition for the ultrasonic ultrafast plane wave data; step three, searching a curvature inflection point for a rank-n tensor curve obtained by the CP decomposition, and determining whether the curvature inflection point satisfies a preset constraint condition; and executing step four when yes, otherwise executing step two; step four, truncating a low-rank signal before the curvature inflection point in the rank-n tensor curve, and reconstructing IQ signal data; and step five, filtering the IQ signal data and performing a fixed delay correlation calculation on the IQ signal data filtered, so as to obtain an ultrasonic Doppler blood flow signal. By adopting the method of matrix decomposition in the present disclosure, the three-dimensional data is converted into tensors and the rank decomposition is directly performed; the decomposed low rank is truncated to filter the noise; and the fixed delay correlation calculation is performed to obtain the ultrasonic blood flow information.

### CROSS-REFERENCE TO RELATED APPLICATION

The present disclosure claims priority to Chinese patent application No. 2023109588056, filed with the Chinese Patent Office on Aug. 1, 2023, entitled “ULTRASONIC MICRO BLOOD FLOW IMAGING METHOD BASED ON RANK DECOMPOSITION FILTERING”, the entire contents of which are incorporated herein by reference.

### TECHNICAL FIELD

The present disclosure relates to the technology field of ultrasonic imaging, and in particular to an ultrasonic micro blood flow imaging method based on rank decomposition filtering.

### BACKGROUND ART

At present, the ultrasonic micro blood flow imaging is a high-resolution ultrasonic technique, wherein the imaging result can be used to assess a blood flow status of micro-vessels in human tissues. It can provide detailed information about the blood flow velocity, volume, and direction of the micro-vessels, which has important implications for the diagnosis and treatment of many diseases. In conjunction with the advantages of ultrasound, such as non-invasive, non-invasive, and non-radiation, the ultrasonic micro blood flow imaging has been widely used in clinics. The common clinical application scenarios of the ultrasonic micro blood flows include, for example, tumor assessment, cardiovascular disease detection, providing detailed information on cardiac function and myocardial contraction, brain neurology application, and assessment of human microcirculation. In general, the ultrasonic micro blood flow imaging has been used in many medical fields, including oncology, cardiovascular, neurology, and microcirculation, etc. It provides detailed blood flow information, which can help the doctor diagnose diseases more accurately, formulate treatment plans, and monitor treatment effects, and son.

Currently, the most widely used filtering method in the ultrasonic micro blood flow imaging is singular value decomposition (SVD). It is different from the infinite impulse response (IIR) or finite impulse response (FIR) filtering in the traditional ultrasonic blood flow imaging technique, where the SVD filter combines and converts the spatial-temporal information of the signal into the product of three matrixes instead of converting the time domain signal to the frequency domain for frequency blocking, which means that one matrix A is represented as the product of U, Σ, and V, where U and V are orthogonal matrixes, and Σ is the diagonal matrix. The singular value extraction and signal denoising are performed by using the features in the diagonal matrix, and a formula of the singular value decomposition is as follows:

A=UΣV^{T},

where U is a matrix with a dimension of m×m; Σ is a matrix with a dimension of m×n; all elements except those on the main diagonal are zero, and each element on the main diagonal is referred to as a singular value; and V is a matrix with a dimension of n×n, as shown in FIG. 1.

The existing Chinese patent CN115969413A discloses an ultrasonic color blood flow filtering method based on singular value decomposition, which sets different thresholds by comparing the decomposition and reconstruction of the SVD filter with the original signal for determination, so as to dynamically select the range of SVD filtering. Compared with the traditional color signal processing method, it can adaptively determine the most suitable threshold value according to real-time signal characteristics, which can more accurately filter the clutter, so that the blood flow signal is more accurate.

Additionally, for multi-angle plane wave ultrasonic micro blood flow imaging, it needs to process ultrasonic sequence data composed of a duration of a specific length to transform it into a spatiotemporal joint matrix, which converts the three-dimensional matrix into a two-dimensional matrix for SVD filtering. However, in cases where a large amount of data points are collected, the dimension conversion and SVD filtering will consume a considerable amount of time, which causes a large obstacle to the improvement of the real-time frame rate in ultrasonic ultrafast sequences. The common signal processing optimization method generally uses block sampling or random sampling in conjunction with SVD, so as to improve the computational speed of this step. The existing patent CN112914611A discloses an ultrasonic micro-vessel imaging method based on random singular value decomposition and weighted L1 paradigm, which adopts the random singular value decomposition to construct a sparse matrix to improve the external weight of the micro-vessels after randomly sampling the spatiotemporal combined reconstruction matrix, and then optimizes the cost function by using the projected gradient descent method to obtain the ultrasonic micro-vessel imaging result with the addition of a total variation regularization. The method increases the external weight of the micro-vessels to better suppress the noise outside the micro-vessels, which improves the signal to noise ratio (SNR) of the micro-vessel imaging and improves the calculational speed.

It can be seen that the methods used in the prior art have defects such as high complexity of realization and insufficient accuracy of the blood flow information.

### SUMMARY

One of the objectives of the present disclosure is to provide an ultrasonic micro blood flow imaging method based on rank decomposition filtering to solve the problem of the long processing time of SVD filtering in duplex mode under ultrafast imaging. By adopting the method of matrix decomposition, the three-dimensional data is converted into tensors and the rank decomposition is directly performed; the decomposed low rank is truncated to filter the noise; and the fixed delay correlation calculation is performed to obtain the ultrasonic blood flow information.

The embodiments of the present disclosure provide an ultrasonic micro blood flow imaging method based on rank decomposition filtering, including:
step one, acquiring ultrasonic ultrafast plane wave data;
step two, reading the amount of decomposed rank-1 tensors and performing a CP decomposition for the ultrasonic ultrafast plane wave data;
step three, searching a curvature inflection point for a rank-n tensor curve obtained by the CP decomposition; and determining whether the curvature inflection point satisfies a preset constraint condition, and executing step four when yes, otherwise executing step two;
step four, truncating a low-rank signal before the curvature inflection point in the rank-n tensor curve, and reconstructing IQ signal data; and
step five, filtering the IQ signal data and performing a fixed delay correlation calculation on the IQ signal data filtered, so as to obtain an ultrasonic Doppler blood flow signal.

Optionally, a dimension of the ultrasonic ultrafast plane wave data is m×n×t, where m is a number of longitudinal sampling points, n is a number of transverse sampling points, and t is a data length in a slow time direction.

Optionally, a conversion formula between a measured Doppler velocity and a pulse repetition frequency in a slow-time t direction after multi-angle compounding is as follows:

$$PRF = \frac{4}{λ} {Scale}_{max} ,$$

where PRF is the pulse repetition frequency in the slow-time t direction after the multi-angle compounding; Scaleₘₐₓ is a measurement value range of a micro blood flow Doppler signal by a user; and λ is an emission wavelength.

Optionally, in the step two, the amount of the rank-1 tensors is set as a total length of the slow-time t direction, between [20, 128].

Optionally, in the step two, the CP decomposition decomposes the tensor into a sum of rank-1 tensors, which is expressed in a formula as follows:

$$χ \approx {\sum }_{r = 1}^{R} {a}_{r} ∘ {b}_{r} ∘ {c}_{r} ,$$

where ∘ represents taking an outer product; χ is the tensor; aᵣ, bᵣ, and cᵣ represent three vectors of three dimensions respectively; R is a rank in the CP decomposition; and a three-dimensional tensor can be regarded as a sum of outer products of aᵣ, bᵣ, and cᵣ in different ranks.

The tensor is matrixed at the same time, and all a vectors are merged into the A matrix for calculation, so as to obtain A=[a1, a2, . . . , aR], which is extended to the B and C matrixes. The matrixed tensor CP decomposition is expressed as:

X₍₁₎≈A(B)⊙C)^{T},

X₍₂₎≈B(C)⊙A)^{T},

X₍₃₎≈C(B⊙A)^{T},

where ⊙ represents a Khatri-Rao product, and X₍ᵢ₎ represents a matrix obtained by the mode i matricization of the tensor χ.

After the CP decomposition, assuming that column vectors of the matrixes A, B, and C are standardized vectors, an extracted weight is merged into a vector λ∈R, and an expression of the CP decomposition is as follows:

$$X = 〚 λ ; A , B , C 〛 ≡ {\sum }_{r = 1}^{R} {λ}_{r} {a}_{r} ∘ {b}_{r} ∘ {c}_{r} .$$

Optionally, in the step three, when searching a curvature inflection point for a rank-n tensor curve obtained by the CP decomposition, the extracted weight is merged into the vector λ, and a vector curve is extended from a high rank interval to a low rank for curve fitting using a least squares method to find the curvature inflection point.

Optionally, in the step three, the constraint condition is that a minimum value of a curvature inflection point coordinate index is larger than or equal to 2

Optionally, the step of truncating a low-rank signal before the curvature inflection point in the rank-n tensor curve, and reconstructing IQ signal data includes:

truncating the vector λ before the curvature inflection point in the rank-n tensor curve as zero, and reconstructing the IQ signal data according to the CP decomposition expression.

Optionally, a dimension of the IQ signal data is m×n×t.

Optionally, a calculation formula for performing a fixed delay correlation calculation on the IQ signal data filtered is as follows:

$${R}_{1} ( i , j ) = \frac{1}{EL - 1} {\sum }_{k = 1}^{EL - 1} WFIQ ( i , j , k + 1 ) {WFIQ ( i , j , k )}^{*} ,$$

$${R}_{0} ( i , j ) = \frac{1}{EL} {\sum }_{k = 1}^{EL} WFIQ ( i , j , k ) {WFIQ ( i , j , k )}^{*} ,$$

where WFIQ represents IQ data filtered, EL represents the length of Ensemble, that is, the length of the slow-time t direction in the step one, i and j represent coordinates of rows and columns in space respectively, R₀ represents a zero delay correlation, R₁ represents the fixed delay correlation, the fixed delay is taken to be 1, and * is a conjugate complex number symbol, wherein an inverse tangent function calculation and a velocity conversion are performed on the acquired R₁ to obtain a Doppler blood flow velocity signal micro blood flow image, and a modulus calculation and threshold compression are performed on R₀ to obtain a Doppler blood flow intensity signal micro blood flow image.

Other features and advantages of the present disclosure will be illustrated in the subsequent specification, become apparent in part from the specification, or be understood through the implementation of the present disclosure. The objectives and other advantages of the present disclosure may be realized and obtained by structures specifically indicated in the written specification, claims, and drawings.

The technical solutions of the present disclosure are described in further detail below by the drawings and embodiments.

### BRIEF DESCRIPTION OF DRAWINGS

The drawings are used to provide a further understanding of the present disclosure and constitute a part of the specification, which are used in conjunction with the embodiments of the present disclosure to illustrate the present disclosure and do not constitute a limitation of the present disclosure. In the drawings:

FIG. 1 shows a flowchart of an ultrasonic micro blood flow imaging method based on rank decomposition filtering provided by the embodiments of the present disclosure;

FIG. 2 shows a schematic diagram of an SVD decomposition for a matrix A with a dimension of M×N in the embodiments of the present disclosure;

FIG. 3 shows a schematic diagram of a CP decomposition for a tensor χ in the embodiments of the present disclosure; and

FIG. 4 shows curves of rank N of different lengths in the embodiments of the present disclosure.

### DETAILED DESCRIPTION OF EMBODIMENTS

The preferred embodiments of the present disclosure are described below in conjunction with drawings. It should be understood that the preferred embodiments described herein are intended only to illustrate and explain the present disclosure, and are not intended to limit the present disclosure.

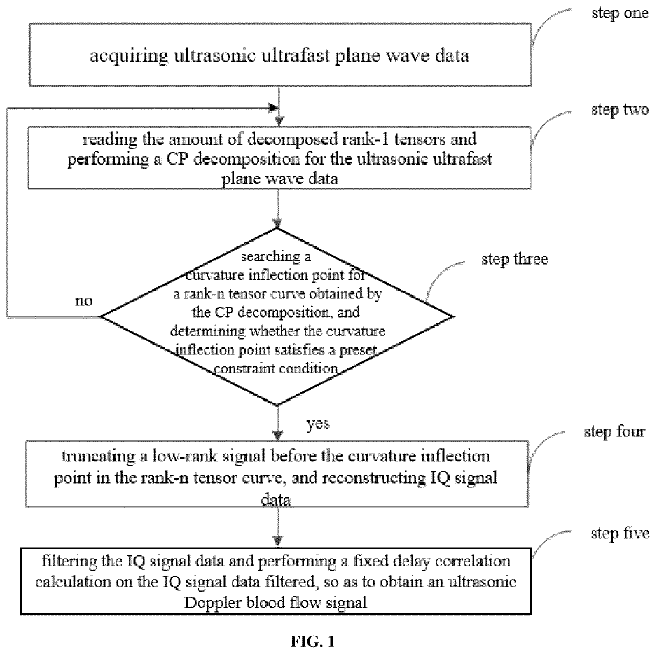

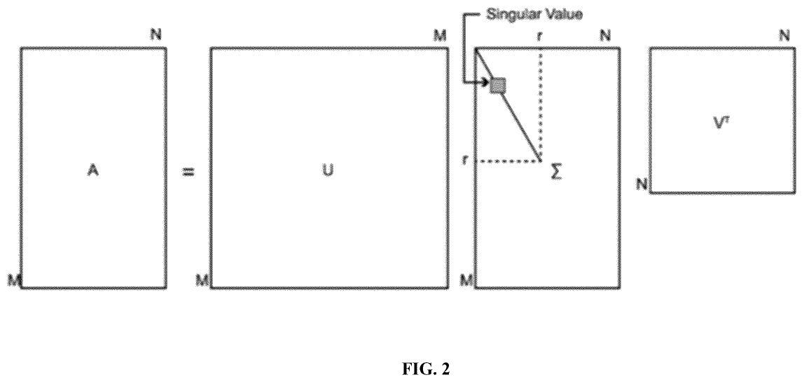

Referring to FIG. 1 and FIG. 2, FIG. 1 shows a flowchart of an ultrasonic micro blood flow imaging method based on rank decomposition filtering provided by the embodiments of the present disclosure, and FIG. 2 shows a schematic diagram of an SVD decomposition for a matrix A with a dimension of M×N in the embodiments of the present disclosure. The method specifically includes the following steps:
step one, acquiring ultrasonic ultrafast plane wave data;
step two, reading the amount of decomposed rank-1 tensors and performing a CP decomposition for the ultrasonic ultrafast plane wave data;
step three, searching a curvature inflection point for a rank-n tensor curve obtained by the CP decomposition, and determining whether the curvature inflection point satisfies a preset constraint condition; and executing step four when yes, otherwise executing step two;
step four, truncating a low-rank signal before the curvature inflection point in the rank-n tensor curve, and reconstructing IQ signal data; and
step five, filtering the IQ signal data and performing a fixed delay correlation calculation on the IQ signal data filtered, so as to obtain an ultrasonic Doppler blood flow signal.

The dimension of the ultrasonic ultrafast plane wave data is m×n×t, where m is a number of longitudinal sampling points, n is a number of transverse sampling points, and t is a data length in a slow time direction.

The conversion formula between a measured Doppler velocity and a pulse repetition frequency in a slow-time t direction after multi-angle compounding is as follows:

$$PRF = \frac{4}{λ} {Scale}_{max} ,$$

where PRF is the pulse repetition frequency in the slow-time t direction after the multi-angle compounding; Scaleₘₐₓ is a measurement value range of a micro blood flow Doppler signal by a user; and λ is an emission wavelength.

In the step two, the amount of the rank-1 tensors is set as a total length of the slow-time t direction, between [20, 128].

In the step two, the CP decomposition decomposes the tensor into a sum of rank-1 tensors, which is expressed in a formula as follows:

$$χ \approx {\sum }_{r = 1}^{R} {a}_{r} ∘ {b}_{r} ∘ {c}_{r} ,$$

where ∘ represents taking an outer product; χ is the tensor; aᵣ, bᵣ, and cᵣ represent three vectors of three dimensions respectively; R is a rank in the CP decomposition; and a three-dimensional tensor can be regarded as a sum of outer products of aᵣ, bᵣ, and cᵣ in different ranks.

The tensor is matrixed, and all a vectors are merged into a A matrix for calculation, so as to obtain A=[a1, a2, . . . , aR], which is extended to the B and C matrixes, and the matrixed tensor CP decomposition is expressed as:

X₍₁₎≈A(B)⊙C)^{T},

X₍₂₎≈B(C)⊙A)^{T},

X₍₃₎≈C(B⊙A)^{T},

where ⊙ represents a Khatri-Rao product, and X₍ᵢ₎ represents a matrix obtained by the mode i matricization of the tensor χ.

After the CP decomposition, assuming that column vectors of the matrixes A, B, and C are standardized vectors, an extracted weight is merged into a vector λ∈R, and an expression of the CP decomposition is as follows:

$$X \approx \text{?} λ ; A , B , C \text{?} ≡ {\sum }_{r = 1}^{R} {λ}_{r} {a}_{r} ∘ {b}_{r} ∘ {c}_{r} .$$

In the step three, when searching the curvature inflection point for the rank-n tensor curve obtained by the CP decomposition, the extracted weight is merged into the vector λ, and a vector curve is extended from a high rank interval to a low rank for curve fitting using a least squares method to find the curvature inflection point.

In the step three, the constraint condition is that the minimum value of a curvature inflection point coordinate index is larger than or equal to 2.

In step four, the step of truncating a low-rank signal before the curvature inflection point in the rank-n tensor curve, and reconstructing IQ signal data includes:

truncating the vector λ before the curvature inflection point in the rank-n tensor curve as zero, and reconstructing the IQ signal data according to the CP decomposition expression.

The dimension of the IQ signal data is also m×n×t.

The calculation formula for performing a fixed delay correlation calculation on the IQ signal data filtered is as follows:

$${R}_{1} ( i , j ) = \frac{1}{EL - 1} {\sum }_{k = 1}^{EL - 1} WFIQ ( i , j , k + 1 ) {WFIQ ( i , j , k )}^{*} ,$$

$${R}_{0} ( i , j ) = \frac{1}{EL} {\sum }_{k = 1}^{EL} WFIQ ( i , j , k ) {WFIQ ( i , j , k )}^{*} ,$$

where WFIQ represents IQ data filtered, EL represents the length of Ensemble, that is, the length of the slow-time t direction in the step one, i and j represent coordinates of rows and columns in space respectively, R₀ represents a zero delay correlation, R₁ represents the fixed delay correlation, the fixed delay is taken to be 1, and * is a conjugate complex number symbol, wherein an inverse tangent function calculation and a velocity conversion are performed on the acquired R₁ to obtain a Doppler blood flow velocity signal micro blood flow image, and a modulus calculation and threshold compression are performed on R₀ to obtain a Doppler blood flow intensity signal micro blood flow image.

The following describes a specific realization method of the ultrasonic micro blood flow imaging method based on the rank decomposition filtering provided by the embodiments of the present disclosure.

The ultrasonic ultrafast plane wave data compounded from multiple angles is acquired, with a dimension of m×n×t, where m is a number of longitudinal sampling points, n is a number of transverse sampling points, and t is a data length in a slow time direction. For the pulse repetition frequency (PRF) in the slow-time t direction after the multi-angle compounding, a conversion formula between the measured Doppler velocity and the PRF is:

$$PRF = \frac{4}{λ} {Scale}_{max} ,$$

where Scaleₘₐₓ is a measurement value range of a micro blood flow Doppler signal by a user, and λ is an emission wavelength.

The amount of decomposed rank-1 tensors is read from the preset parameters and the CP decomposition is performed on the three-dimensional data.

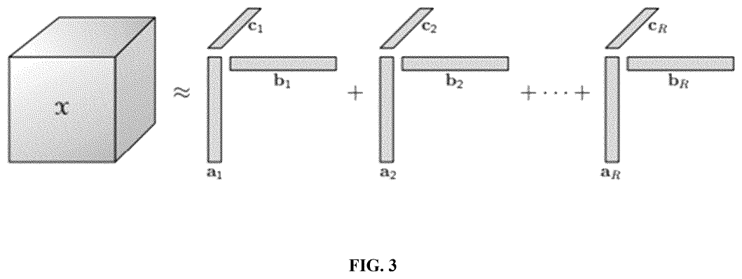

The amount of the preset rank-1 tensors is set as a total length of the slow-time t direction, generally between [20, 128]. The CP decomposition was first proposed by F. L. Hitchcock in The Expression of a Tensor or a Polyadic as a Sum of Products, F. L. Hitchcock, 1927, Journal of Mathematics and Physics. It is essentially to decompose the tensor into a sum of rank-1 tensors, and the formula can be expressed as:

$$χ \approx {\sum }_{r = 1}^{R} {a}_{r} ∘ {b}_{r} ∘ {c}_{r} ,$$

where ∘ represents taking an outer product; χ is the tensor; aᵣ, bᵣ, and cᵣ represent three vectors of three dimensions respectively; R is a rank in the CP decomposition; and a three-dimensional tensor can be regarded as a sum of outer products of aᵣ, bᵣ, and cᵣ in different ranks. The schematic diagram of the CP decomposition is shown in FIG. 3.

The tensor can be matrixed at the same time. All a vectors are merged into an A matrix for calculation, so as to obtain A=[a1, a2, . . . , aR], and this theory can be expanded to the B and C matrixes. Therefore, the matrixed tensor CP decomposition is expressed as:

X₍₁₎≈A(B)⊙C)^{T},

X_{(Z)}≈B(C)⊙A)^{T},

X₍₃₎≈C(B⊙A)^{T},

where ⊙ represents a Khatri-Rao product, and X₍ᵢ₎ represents a matrix obtained by the mode i matricization of the tensor χ.

Generally, after the CP decomposition, assuming that column vectors of the matrixes A, B, and C are standardized vectors, an extracted weight is merged into a vector λ∈R. Finally, an expression of the CP decomposition can be as follows:

$$X \approx \text{?} λ ; A , B , C \text{?} ≡ {\sum }_{r = 1}^{R} {λ}_{r} {a}_{r} ∘ {b}_{r} ∘ {c}_{r} .$$

A curvature inflection point is sought for the rank-n tensor curve obtained by decomposition. If the curvature inflection point does not satisfy the preset constraint condition, the amount of rank-n tensors is increased, and the step two is repeated.

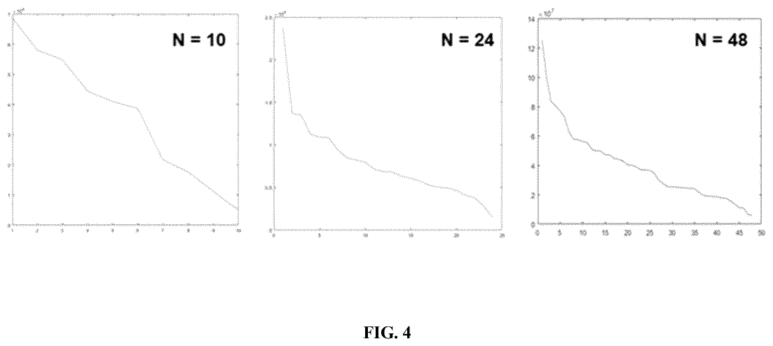

The extracted weight is merged into the vector λ, and a vector curve is extended from a high rank interval to a low rank for curve fitting using a least squares method to find the curvature inflection point. The minimum value of a curvature inflection point coordinate index is larger than or equal to 2. If the constraint condition is not satisfied, the length k is increased to obtain a rank n+k of the CP decomposition, and step two is repeated until the curvature inflection point coordinate index p is found and satisfies the constraint condition, wherein k in the present disclosure can be 12. FIG. 4 describes rank-N curves of different lengths, where N=10, N=24, and N=48 respectively, and the vertical axis is the magnitude of the extracted weights.

The low-rank signal before the selected curvature inflection point is truncated, and the three-dimensional IQ signal is reconstructed, so as to obtain data with the same dimensions m×n×t.

The vector λ before the curvature inflection point is truncated to 0, and the three-dimensional IQ signal is reconstructed according to the above CP decomposition expression.

The fixed delay correlation calculation is performed on the IQ data filtered to obtain the ultrasonic Doppler blood flow signal, and the delay correlation calculation is as follows:

$${R}_{1} ( i , j ) = \frac{1}{EL - 1} {\sum }_{k = 1}^{EL - 1} WFIQ ( i , j , k + 1 ) {WFIQ ( i , j , k )}^{*} ,$$

$${R}_{0} ( i , j ) = \frac{1}{EL} {\sum }_{k = 1}^{EL} WFIQ ( i , j , k ) {WFIQ ( i , j , k )}^{*} ,$$

where WFIQ represents IQ data filtered, EL represents a length of Ensemble, that is, a length of the slow-time t direction in the step one, i and j represent coordinates of rows and columns in a space respectively, R0 represents a zero delay correlation, R1 represents the fixed delay correlation, the fixed delay in the present disclosure is taken to be 1, and * is a conjugate complex number symbol. An inverse tangent function calculation and a velocity conversion are performed on the acquired R₁ to obtain a Doppler blood flow velocity signal micro blood flow image, and a modulus calculation and threshold compression are performed on R₀ to obtain a Doppler blood flow intensity signal micro blood flow image.

In summary, the present disclosure provides the ultrasonic micro blood flow imaging method based on rank decomposition filtering to solve the problem of the long processing time of SVD filtering in duplex mode under ultrafast imaging. By adopting the method of matrix decomposition, the three-dimensional data is converted into tensors and the rank decomposition is directly performed; the decomposed low rank is truncated to filter the noise; and the fixed delay correlation calculation is performed to obtain the ultrasonic blood flow information. Compared to the prior art, it reduces the realization complexity and improves the accuracy of the obtained ultrasonic blood flow information.

Additionally, the embodiments of the present disclosure provide an ultrasonic micro blood flow imaging device based on rank decomposition filtering, wherein the device can perform the method described in any one of the realization methods in the above embodiments. The device can include an acquisition module, a decomposition module, a detection module, a reconstruction module, and a calculation module.

The acquisition module is configured to acquire ultrasonic ultrafast plane wave data.

The decomposition module is configured to read the amount of decomposed rank-1 tensors and perform a CP decomposition for the ultrasonic ultrafast plane wave data.

The detection module is configured to search a curvature inflection point for a rank-n tensor curve obtained by the CP decomposition, and determine whether the curvature inflection point satisfies a preset constraint condition.

The reconstruction module is configured to truncate a low-rank signal before the curvature inflection point in the rank-n tensor curve and reconstruct IQ signal data, when the detection result of the detection module is yes.

The calculation module is configured to filter the IQ signal data and perform a fixed delay correlation calculation on the IQ signal data filtered, so as to obtain an ultrasonic Doppler blood flow signal.

In one possible realization method, the dimension of the ultrasonic ultrafast plane wave data is m×n×t, where m is the number of longitudinal sampling points, n is the number of transverse sampling points, and t is the data length of the slow time direction.

In one possible realization method, a conversion formula between a measured Doppler velocity and a pulse repetition frequency in a slow-time t direction after multi-angle compounding is as follows:

$$PRF = \frac{4}{λ} {Scale}_{max} ,$$

where PRF is the pulse repetition frequency in the slow-time t direction after the multi-angle compounding; Scaleₘₐₓ is a measurement value range of a micro blood flow Doppler signal by a user; and λ is an emission wavelength.

The technical effects of the ultrasonic micro blood flow imaging device based on rank decomposition filtering provided by the embodiment can be referred to as the technical effects of the method described in any one of the above embodiments, and will not be repeated herein.

Additionally, the embodiments of the present disclosure further provide an electronic device, wherein the electronic device can be a computer device, a server, etc., and the electronic device includes a storage medium and a processor. The components of the storage media and the processor are electrically connected to each other directly or indirectly, so as to realize the transmission or interaction of data. For example, these components can be electrically connected to each other via one or more communication buses or signal lines.

The processor is configured to read/write data or programs stored in the storage medium and to execute the ultrasonic micro blood flow imaging method based on rank decomposition filtering provided by any one of the embodiments of the present disclosure.

The technical effects of the electronic device described in the embodiment can be referred to as the technical effects of the method described in any of the above embodiments, and will not be repeated herein.

The embodiment further provides a computer readable storage medium, and the computer readable storage medium stores the machine-executable instructions, wherein the machine-executable instructions are executed to realize the ultrasonic micro blood flow imaging method based on rank decomposition filtering provided by the above embodiments.

The technical effects of the computer readable storage medium described in the embodiment can be referred to as the technical effects of the method described in any one of the above embodiments, and will not be repeated herein.

Obviously, those skilled in the art can make various changes and variations to the present disclosure without departing from the spirit and scope of the present disclosure. Thus, if these modifications and variations of the present disclosure fall within the scope of the claims of the present disclosure and their equivalents, the present disclosure is also intended to encompass these modifications and variations.

### INDUSTRIAL APPLICABILITY

In summary, the present disclosure provides an ultrasonic micro blood flow imaging method based on rank decomposition filtering, wherein the three-dimensional data is converted into tensors and the rank decomposition is directly performed by the matrix decomposition method; the decomposed low rank is truncated to filter the noise; and the fixed delay correlation calculation is performed to obtain the ultrasonic blood flow information, which reduces the realization complexity and improves the accuracy of the obtained ultrasonic blood flow information.

1. An ultrasonic micro blood flow imaging method based on rank decomposition filtering, comprising:
step one, acquiring ultrasonic ultrafast plane wave data;
step two, reading an amount of decomposed rank-1 tensors and performing a CP decomposition for the ultrasonic ultrafast plane wave data;
step three, searching a curvature inflection point for a rank-n tensor curve obtained by the CP decomposition, and determining whether the curvature inflection point satisfies a preset constraint condition; and executing step four when yes, otherwise executing the step two;
step four, truncating a low-rank signal before the curvature inflection point in the rank-n tensor curve, and reconstructing IQ signal data; and
step five, filtering the IQ signal data and performing a fixed delay correlation calculation on the IQ signal data filtered, so as to obtain an ultrasonic Doppler blood flow signal.

2. The ultrasonic micro blood flow imaging method based on the rank decomposition filtering according to claim 1, wherein a dimension of the ultrasonic ultrafast plane wave data is m×n×t, where m is a number of longitudinal sampling points, n is a number of transverse sampling points, and t is a data length in a slow time direction.

3. The ultrasonic micro blood flow imaging method based on the rank decomposition filtering according to claim 2, wherein a conversion formula between a measured Doppler velocity and a pulse repetition frequency in a slow-time t direction after multi-angle compounding is as follows:
$$PRF = \frac{4}{λ} {Scale}_{max} ,$$
where PRF is the pulse repetition frequency in the slow-time t direction after the multi-angle compounding; Scaleₘₐₓ is a measurement value range of a microfine blood flow Doppler signal by a user by a user; and λ is an emission wavelength.

4. The ultrasonic micro blood flow imaging method based on the rank decomposition filtering according to claim 1, wherein in the step two, the amount of the rank-1 tensors is set as a total length of the slow-time t direction, between [20, 128].

5. The ultrasonic micro blood flow imaging method based on the rank decomposition filtering according to claim 1, wherein in the step two, the CP decomposition decomposes the tensors into a sum of the rank-1 tensors, which is expressed in a formula as follows:
$$χ \approx {\sum }_{r = 1}^{R} {a}_{r} ∘ {b}_{r} ∘ {c}_{r} ,$$
where ∘ represents taking an outer product; χ is the tensor; aᵣ, bᵣ, and cᵣ represent three vectors of three dimensions respectively; R is a rank in the CP decomposition; and a three-dimensional tensor is able to be regarded as a sum of outer products of aᵣ, bᵣ, and cᵣ in different ranks;
the tensor is performed by a matrixization to merge all a vectors into a A matrix for calculation, so as to obtain A=[a1, a2, . . . , aR], which is extended to B and C matrixes, wherein the matrixed tensor CP decomposition is expressed as:
X₍₁₎≈A(B)⊙C)^{T},
X₍₂₎≈B(C)⊙A)^{T},
X₍₃₎≈C(B⊙A)^{T},
where ⊙ represents a Khatri-Rao product, and X₍ᵢ₎ represents a matrix obtained by the mode i matricization of the tensor χ; and
after the CP decomposition, when column vectors of the matrixes A, B, and C are standardized vectors, an extracted weight is merged into a vector λ∈R, and an expression of the CP decomposition is as follows:
$$X \approx \text{?} λ ; A , B , C \text{?} ≡ {\sum }_{r = 1}^{R} {λ}_{r} {a}_{r} ∘ {b}_{r} ∘ {c}_{r} .$$

6. The ultrasonic micro blood flow imaging method based on the rank decomposition filtering according to claim 5, wherein in the step three, when searching a curvature inflection point for a rank-n tensor curve obtained by the CP decomposition, the extracted weight is merged into the vector λ, and a vector curve is extended from a high rank interval to a low rank for curve fitting using a least squares method to find the curvature inflection point.

7. The ultrasonic micro blood flow imaging method based on the rank decomposition filtering according to claim 1, wherein in the step three, the constraint condition is that a minimum value of a curvature inflection point coordinate index is larger than or equal to 2.

8. The ultrasonic micro blood flow imaging method based on the rank decomposition filtering according to claim 5, wherein the step of truncating a low-rank signal before the curvature inflection point in the rank-n tensor curve, and reconstructing IQ signal data comprises:
truncating the vector λ before the curvature inflection point in the rank-n tensor curve as zero, and reconstructing the IQ signal data according to the CP decomposition expression.

9. The ultrasonic micro blood flow imaging method based on the rank decomposition filtering according to claim 1, wherein a dimension of the IQ signal data is m×n×t.

10. The ultrasonic micro blood flow imaging method based on the rank decomposition filtering according to claim 2, wherein a calculation formula of performing a fixed delay correlation calculation on the IQ signal data filtered is as follows:
$${R}_{1} ( i , j ) = \frac{1}{EL - 1} {\sum }_{k = 1}^{EL - 1} WFIQ ( i , j , k + 1 ) {WFIQ ( i , j , k )}^{*} ,$$
$${R}_{0} ( i , j ) = \frac{1}{EL} {\sum }_{k = 1}^{EL} WFIQ ( i , j , k ) {WFIQ ( i , j , k )}^{*} ,$$
where WFIQ represents IQ data filtered, EL represents a length of Ensemble, that is, a length of the slow-time t direction in the step one, i and j represent coordinates of rows and columns in a space respectively, R₀ represents a zero delay correlation, R₁ represents the fixed delay correlation, and the fixed delay is taken to be 1, and * is a conjugate complex number symbol, wherein an inverse tangent function calculation and a velocity conversion are performed on the acquired R₁ to obtain a Doppler blood flow velocity signal microfine blood flow image, and a modulus calculation and threshold compression are performed on R₀ to obtain a Doppler blood flow intensity signal microfine blood flow image.

11. An ultrasonic micro blood flow imaging device based on rank decomposition filtering, wherein the device comprises:
an acquisition module, configured to acquire ultrasonic ultrafast plane wave data;
a decomposition module, configured to read an amount of decomposed rank-1 tensors and perform a CP decomposition for the ultrasonic ultrafast plane wave data;
a detection module, configured to search a curvature inflection point for a rank-n tensor curve obtained by the CP decomposition, and determine whether the curvature inflection point satisfies a preset constraint condition;
a reconstruction module, configured to truncate a low-rank signal before the curvature inflection point in the rank-n tensor curve and reconstruct IQ signal data, when a detection result of the detection module is yes; and
a calculation module, configured to filter the IQ signal data and perform a fixed delay correlation calculation on the IQ signal data filtered, so as to obtain an ultrasonic Doppler blood flow signal.

12. The ultrasonic micro blood flow imaging device based on the rank decomposition filtering according to claim 11, wherein a dimension of the ultrasonic ultrafast plane wave data is m×n×t, where m is a number of longitudinal sampling points; n is a number of transverse sampling points; and t is a data length of a slow time direction.

13. The ultrasonic micro blood flow imaging device based on the rank decomposition filtering according to claim 11, wherein a conversion formula between a measured Doppler velocity and a pulse repetition frequency in a slow-time t direction after multi-angle compounding is as follows:
$$PRF = \frac{4}{λ} {Scale}_{max} ,$$
where PRF is the pulse repetition frequency in the slow-time t direction after the multi-angle compounding; Scaleₘₐₓ is a measurement value range of a microfine blood flow Doppler signal by a user; and A is an emission wavelength.

14. An electronic device, comprising one or more storage media and one or more processors in communication with the storage media, wherein the one or more storage media store machine-executable instructions executable by the processors, and when the electronic device is in operation, the processors execute the machine-executable instructions to perform the method according to claim 1.

15. A non-transitory computer readable storage medium, wherein the non-transitory computer readable storage medium stores machine-executable instructions, and the method according to claim 1 is realized when machine-executable instructions are executed by a processor.

16. The electronic device according to claim 14, wherein a dimension of the ultrasonic ultrafast plane wave data is m×n×t, where m is a number of longitudinal sampling points, n is a number of transverse sampling points, and t is a data length in a slow time direction.

17. The electronic device according to claim 16, wherein a conversion formula between a measured Doppler velocity and a pulse repetition frequency in a slow-time t direction after multi-angle compounding is as follows:
$$PRF = \frac{4}{λ} {Scale}_{max} ,$$
where PRF is the pulse repetition frequency in the slow-time t direction after the multi-angle compounding; Scaleₘₐₓ is a measurement value range of a microfine blood flow Doppler signal by a user by a user; and A is an emission wavelength.

18. The non-transitory computer readable storage medium according to claim 15, wherein a dimension of the ultrasonic ultrafast plane wave data is m×n×t, where m is a number of longitudinal sampling points, n is a number of transverse sampling points, and t is a data length in a slow time direction.

19. The non-transitory computer readable storage medium according to claim 18, wherein a conversion formula between a measured Doppler velocity and a pulse repetition frequency in a slow-time t direction after multi-angle compounding is as follows:
$$PRF = \frac{4}{λ} {Scale}_{max} ,$$
where PRF is the pulse repetition frequency in the slow-time t direction after the multi-angle compounding; Scaleₘₐₓ is a measurement value range of a microfine blood flow Doppler signal by a user by a user; and A is an emission wavelength.

20. The non-transitory computer readable storage medium according to claim 15, wherein in the step two, the amount of the rank-1 tensors is set as a total length of the slow-time t direction, between [20, 128].
